(19) United States
(12) Patent Application Publication
(10) Pub. No.: US 2026/0014282 A1
(43) Pub. Date: Jan. 15, 2026

(54) METHODS OF FORMING RADIOPAQUE PEPTIDES FOR USE IN MEDICAL HYDROGELS

(71) Applicant: Boston Scientific Scimed, Inc., Maple Grove, MN (US)

(72) Inventors: Rick William Dorn, Plymouth, MN (US); Yen-Hao Hsu, Shrewsbury, MA (US); Cristian Parisi, Amherst, MA (US); Lauren Sfakis Lydecker, Upton, MA (US); Philip David Dorgan, Cork (IE)

(73) Assignee: Boston Scientific Scimed, Inc., Maple Grove, MN (US)

(21) Appl. No.: 19/268,239

(22) Filed: Jul. 14, 2025

Related U.S. Application Data
(60) Provisional application No. 63/671,482, filed on Jul. 15, 2024.

(51) Int. Cl. A61K 49/04; C07K 1/06; C07K 1/10; C07K 7/06

(52) U.S. Cl. CPC A61K 49/0442; C07K 1/062; C07K 1/063; C07K 1/10; C07K 7/06

(57) ABSTRACT

In various aspects, the present disclosure provides methods for forming radiopaque peptides that comprise one or more iodinated amino acid residues and one or more amine-based amino acid residues. Typically, the peptides contain from 3 to 20 amino acid residues. The present disclosure relates to methods of forming such radiopaque peptides, to the use of such radiopaque peptides as crosslinking agents for forming hydrogels, and to hydrogels formed from such radiopaque peptides. The radiopaque peptides and hydrogels are useful, for example, in various medical applications.

### CROSS-REFERENCE TO RELATED APPLICATIONS

This application claims the benefit of U.S. Provisional Patent Application Ser. No. 63/671,482 filed on Jul. 15, 2024, the disclosure of which is incorporated herein by reference.

### FIELD

The present disclosure relates to methods of forming radiopaque peptides, to the use of such radiopaque peptides as crosslinking agents for forming hydrogels, and to hydrogels formed from such radiopaque peptides. The radiopaque peptides and hydrogels are useful, for example, in various medical applications.

### BACKGROUND

SpaceOAR®, a rapid crosslinking hydrogel that polymerizes in vivo within seconds, is based on a multi-arm polyethylene glycol (PEG) polymer functionalized with succinimidyl glutarate as activated end groups which further react with trilysine to form crosslinks. This product has become a very successful, clinically-used biomaterial in prostate cancer therapy. A further improvement based on this structure is that a portion the succinimidyl glutarate end groups have been functionalized with 2,3,5-triiiodobenzamide groups, providing radiopacity. This hydrogel, known by the trade name of SpaceOAR Vue®, is the radiopaque version of SpaceOAR® for prostate medical applications.

Alternative strategies for forming iodine-labelled crosslinked hydrogels that provide enhanced radiopacity while maintaining crosslink density per polymer molecule are of interest for medical applications.

### SUMMARY

The present disclosure pertains to methods of forming iodinated peptide compounds.

In some aspects, the present disclosure pertains to methods for forming radiopaque peptides that comprise: (a) performing an amide coupling reaction between (i) a partially protected first iodinated amino acid comprising an unprotected first α-carbon amino group, a protected first α-carbon carboxylic acid group that is protected with a first carboxylic acid protective group and an iodinated aromatic group and (ii) a partially protected first amine-based amino acid comprising an unprotected second α-carbon carboxylic acid group, a protected second α-carbon amino group that is protected with a first amino protective group, and a protected first side chain amino group that is protected with a second amino protective group that differs from the first amino protective group, is deprotected under different conditions from those used to deprotect the first amino protective group, and is stable under conditions used to deprotect the first amino group, thereby forming an amide linkage from the unprotected first α-carbon amino group and the unprotected second α-carbon carboxylic acid group; (b) deprotecting the protected second α-carbon amino group of the product of step (a) to form a deprotected second α-carbon amino group, while leaving the protected first side chain amino group and the protected first α-carbon carboxylic acid group of the product of step (a) protected; (c) performing an amide coupling reaction between the deprotected second α-carbon amino group of the product of step (b) and a partially protected third amino acid comprising a protected third α-carbon amino group that is protected with the first amino protective group and an unprotected third α-carbon carboxylic acid group, thereby forming an amide linkage from the deprotected second α-carbon amino group and the unprotected third α-carbon carboxylic acid group; (d) deprotecting the protected third α-carbon amino group in the product of step (c) to form a deprotected third α-carbon amino group, while leaving all other protected groups in the product of step (c) protected; (c) optionally repeating steps (c) and (d) with one or more partially protected further amino acids comprising a protected additional α-carbon amino group that is protected with the first amino protective group and an unprotected additional α-carbon carboxylic acid group until a target radiopaque peptide length is achieved; and (f) after the target radiopaque peptide length is achieved, removing all protective groups.

In some aspects, the present disclosure pertains to methods for forming radiopaque peptides that comprise: (a) performing an amide coupling reaction between (i) a partially protected first iodinated amino acid comprising an protected first α-carbon amino group that is protected with a first amino protective group, an unprotected first α-carbon carboxylic acid group and an iodinated aromatic group and (ii) a partially protected first amine-based amino acid comprising a protected second α-carbon carboxylic acid group that is protected with a first carboxylic acid protective group, an unprotected second α-carbon amino group, and a protected first side chain amino group that is protected with a second amino protective group that differs from the first amino protective group, is deprotected under different conditions from those used to deprotect the first amino protective group, and is stable under conditions used to deprotect the first amino group, thereby forming an amide linkage from the unprotected first α-carbon carboxylic acid group and the unprotected second α-carbon amino group; (b) deprotecting the protected first α-carbon amino group in the product of step (a) to form a deprotected first α-carbon amino group, while leaving all other protected groups in the product of step (a) protected while leaving the protected first side chain amino group and the protected second α-carbon carboxylic acid group of the product of step (a) protected; (c) performing an amide coupling reaction between the deprotected first α-carbon amino group of the product of step (b) and a partially protected third amino acid comprising a protected third α-carbon amino group that is protected with the first amino protective group and an unprotected third α-carbon carboxylic acid group, thereby forming an amide linkage from the deprotected first α-carbon amino group and the unprotected third α-carbon carboxylic acid group; (d) deprotecting the protected third α-carbon amino group in the product of step (c) to form a deprotected third α-carbon amino group, while leaving all other protected groups in the product of step (c) protected; (c) optionally repeating steps (c) and (d) with one or more partially protected further amino acids comprising a protected additional α-carbon amino group that is protected with the first amino protective group and an unprotected additional α-carbon carboxylic acid group until a target radiopaque peptide length is achieved; and (f) after the target radiopaque peptide length is achieved, removing all protective groups.

In some embodiments, which can be used in conjunction with any of the above aspects, the partially protected third amino acid is a partially protected second amine-based amino acid that comprises the protected third α-carbon amino group, the unprotected third α-carbon carboxylic acid group, and a protected second side chain amino group that is protected with the second amino protective group

In some embodiments, which can be used in conjunction with any of the above aspects, the partially protected third amino acid is a partially protected second iodinated amino acid that comprises the protected third α-carbon amino group, the unprotected third α-carbon carboxylic acid group, and a second iodinated aromatic group.

In some embodiments, which can be used in conjunction with any of the above aspects, the partially protected third amino acid is a partially protected carboxylic-acid-based amino acid that comprises the protected third α-carbon amino group, the unprotected third α-carbon carboxylic acid group, and a side chain carboxylic acid group that is protected with the first carboxylic acid protective group. In some embodiments, the carboxylic-acid-based amino acid is selected from aspartic acid and glutaric acid.

In some embodiments, which can be used in conjunction with any of the above aspects and embodiments, the first carboxylic acid protective group is a C₁-C₆ alkyl group.

In some embodiments, which can be used in conjunction with any of the above aspects and embodiments, the first carboxylic acid protective group is deprotected by saponification.

In some embodiments, which can be used in conjunction with any of the above aspects and embodiments, the first amino protective group is selected from an acid labile protecting group, such as a tert-butoxycarbonyl (t-Boc) group, a tritylamine group or a benzylideneamine group. In some of these embodiments, the first amino protective group is deprotected by hydrolysis with an acid.

In some embodiments, which can be used in conjunction with any of the above aspects and embodiments, the second amino protective group is an amino protective group that is stable in an acidic environment, such as a 9-fluorenylmethoxycarbonyl (Fmoc) group, an acetamide group, a trifluoroacetamide group, or a benzyl carbamate group. In some of these embodiments, the second amino protective group is deprotected by treatment with a primary or secondary amine or by treatment with basic conditions in an aqueous environment.

In some embodiments, which can be used in conjunction with any of the above aspects and embodiments, the first iodinated amino acid is selected from thyroxine, diiodotryosine, and iodophenyl alanine.

In some embodiments, which can be used in conjunction with any of the above aspects and embodiments, the first amine-based amino acid is selected from lysine and ornithine.

In some embodiments, which can be used in conjunction with any of the above aspects and embodiments, the target peptide length is between 3 and 20 amino acids.

In some embodiments, which can be used in conjunction with any of the above aspects and embodiments, the target peptide length is between 4 and 8 amino acids.

An advantage of the preceding methods is that iodinated peptides can be formed which contain residues of iodinated amino acids, amine-based amino acids, and other amino acids, any essentially any desired sequence and/or ratio.

The above and other aspects, embodiments, features and benefits of the present disclosure will be readily apparent from the following detailed description.

### BRIEF DESCRIPTION OF THE DRAWINGS

FIG. 1 schematically illustrates a processes for forming an iodinated amino acid, in accordance with an embodiment of the present disclosure.

FIGS. 2A-2D schematically illustrate a processes for forming a radiopaque pentapeptide, in accordance with an embodiment of the present disclosure.

FIGS. 3A-3D schematically illustrate a processes for forming a radiopaque pentapeptide, in accordance with another embodiment of the present disclosure.

FIGS. 4A-4E schematically illustrate a processes for forming a radiopaque hexapeptide, in accordance with an embodiment of the present disclosure.

FIG. 5 illustrates a delivery device, in accordance with an embodiment of the present disclosure.

FIG. 6 illustrates a delivery device, in accordance with another embodiment of the present disclosure.

### DETAILED DESCRIPTION

In various aspects, the present disclosure provides methods for forming radiopaque peptides that comprise one or more iodinated amino acid residues and one or more amine-based amino acid residues. Typically, the peptides contain from 3 to 20 amino acid residues, for example, ranging from 3 to 4 to 5 to 6 to 8 to 10 to 12 to 15 to 20 amino acid resides (i.e., ranging between any two of the preceding values).

Examples of amine-based amino acids include 2,6-diaminohexanoic acid (lysine), 2,5-diaminohexanoic acid, 2,4-diaminohexanoic acid, 2,3-diaminohexanoic acid, 2,5-diaminopentanoic acid (ornithine), 2,4-diaminopentanoic acid, 2,3-diaminopentanoic acid, 2,4-diaminobutanoic acid, 2,3-diaminobutanoic acid, 2,3-diaminopropanoic acid (3-amino alanine), and diaminobenzeneacetic acid among others.

An iodinated amino acid is an amino acid in which the side group contains one or more iodine atoms. In various embodiments, the side group of the iodinated amino acid comprises one, two, three, or more iodinated aromatic groups. Examples of iodinated aromatic groups include iodine-substituted monocyclic aromatic groups and iodine-substituted multicyclic aromatic groups, such as, for example, iodine-substituted phenyl groups and iodine-substituted naphthyl groups. The aromatic groups may be substituted with one, two, three, four, five, six or more iodine atoms.

In various embodiments, the aromatic groups may be further substituted with one or more hydrophilic groups, for example, one, two, three, four, five, six or more hydrophilic groups. The hydrophilic groups may comprise, for example, one or more of the following groups: hydroxyl groups, hydroxyalkyl groups (e.g., hydroxyalkyl groups containing one carbon, two carbons, three carbons, four carbons, etc.), and/or ester groups (e.g., ester groups containing two carbons, three carbons, four carbons, five carbons, six carbons, etc.), among others. The hydrophilic groups may be linked to the aromatic group directly or through any suitable linking moiety, which may be selected, for example, from alkyl groups (e.g., alkyl groups containing one carbon, two carbons, three carbons, four carbons, etc.), amide groups, ether groups, ester groups, urea groups, or urethane groups, among others.

In certain embodiments, the iodinated amino acids include amino acids that comprise one, two, three or more hydroxy-iodo-aromatic groups, such as hydroxy-iodo-phenyl groups or hydroxy-iodo-naphthyl groups. The hydroxy-iodo-aromatic groups may be selected, for example, from mono-hydroxy-mono-iodo-phenyl groups, mono-hydroxy-di-iodo-phenyl groups, mono-hydroxy-tri-iodo-phenyl groups, mono-hydroxy-tetra-iodo-phenyl groups, di-hydroxy-mono-iodo-phenyl groups, di-hydroxy-di-iodo-phenyl groups, di-hydroxy-tri-iodo-phenyl groups, tri-hydroxy-mono-iodo-phenyl groups, and tri-hydroxy-di-iodo-phenyl groups, among others.

A few specific examples of iodine-containing amino acids for use in the present disclosure include the following: iodo-phenylalanine,

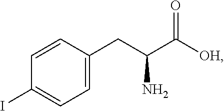 [SMILES: N[C@@H](Cc1ccc(I)cc1)C(=O)O]

which comprises a mono-iodo-phenyl group, monoiodotyrosine,

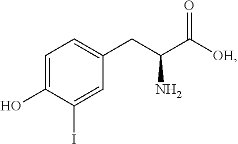 [SMILES: N[C@@H](Cc1ccc(O)c(I)c1)C(=O)O]

which comprises a mono-iodo-phenyl group, specifically, a mono-hydroxy-mono-iodo-phenyl group, diiodotyrosine,

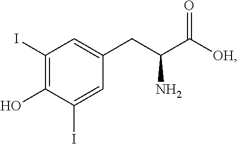 [SMILES: N[C@@H](Cc1cc(I)c(O)c(I)c1)C(=O)O]

which comprises a mono-hydroxy-di-iodo-phenyl group, diiodothyronine,

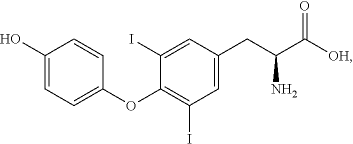 [SMILES: N[C@@H](Cc1cc(I)c(Oc2ccc(O)cc2)c(I)c1)C(=O)O]

which comprises a di-iodo-phenyl group and a hydroxy-phenyl group, triiodothyronine also known as T3,

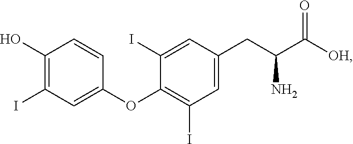 [SMILES: N[C@@H](Cc1cc(I)c(Oc2ccc(O)c(I)c2)c(I)c1)C(=O)O]

which comprises a di-iodo-phenyl group and a mono-hydroxy-mono-iodo-phenyl group, tetraiodothyronine also known as thyroxine or T4,

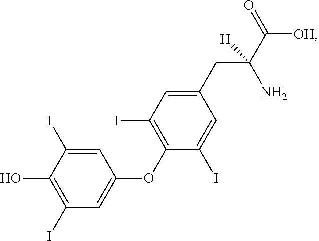 [SMILES: N[C@@H](Cc1cc(I)c(Oc2cc(I)c(O)c(I)c2)c(I)c1)C(=O)O]

which comprises a di-iodo-phenyl group and a mono-hydroxy-di-iodo-phenyl group, iodo-phenylalanine, and iodo-L-DOPA, which comprises a di-hydroxy-mono-iodo-phenyl group, among others. Several iodinated amino acids, along with their CAS numbers, are provided in the following table:

| Name | CAS No. | # I Atoms |
|---|---|---|
| 3-Iodo-L-tyrosine | 70-78-0 | 1 |
| Triiodothyronine | 6893-02-3 | 3 |
| Thyroxine | 51-48-9 | 4 |
| Diiodothyronine | 1041-01-6 | 2 |
| 4-Iodo-L-phenylalanine | 24250-85-9 | 1 |
| 3,5-Diiodotyrosine | 66-02-4 | 2 |
| 4-Iodo-D-phenylalanine | 62561-75-5 | 1 |
| 3,3′-Diiodo-L-thyronine | 4604-41-5 | 2 |
| 3′,5′,3-Triiodothyronine | 5817-39-0 | 3 |

Further iodinated amino acid derivatives can be synthesized via reaction of an aromatic iodinated species with an amine or carboxylic acid side group of an amino acid. Examples include the primary amine-based amino acids discussed above and carboxylic-acid-based amino acids such as aspartic acid and glutaric acid, among others.

Carboxylic-acid-containing iodinated compound for use in the present disclosure include triiodobenzoic acid

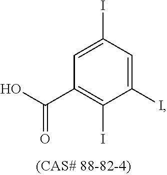 [SMILES: O=C(O)c1cc(I)cc(I)c1I]

diatrizoic acid,

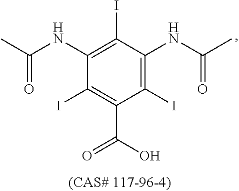 [SMILES: CC(=O)Nc1c(I)c(NC(C)=O)c(I)c(C(=O)O)c1I]

N-acetyl-3,5-diiodo-L-tyrosine,

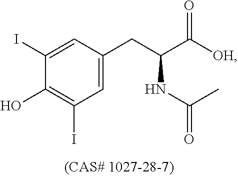 [SMILES: CC(=O)N[C@@H](Cc1cc(I)c(O)c(I)c1)C(=O)O]

N-acetyl-3-diiodo-L-tyrosine,

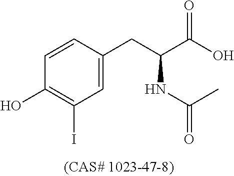 [SMILES: CC(=O)N[C@@H](Cc1ccc(O)c(I)c1)C(=O)O]

and N-acetyl-thyroxine,

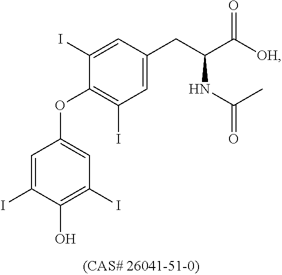 [SMILES: CC(=O)N[C@@H](Cc1cc(I)c(Oc2cc(I)c(O)c(I)c2)c(I)c1)C(=O)O]

among many others.

Various carboxylic-acid-containing iodinated compounds, along with their CAS numbers, are listed in the following table.

| | | # I |
|---|---|---|
| Name | CAS | atoms |
| 2-Iodobenzoic acid | 88-67-5 | 1 |
| 4-Iodobenzoic acid | 619-58-9 | 1 |
| 3-Iodobenzoic acid | 618-51-9 | 1 |
| 4-Iodopicolinic acid | 405939-79-9 | 1 |
| 3,5-Diiodobenzoic acid | 19094-48-5 | 2 |
| 3,4-Diiodobenzoic acid | 35674-20-5 | 2 |
| 3,5-Diiodo-4-(4-methoxyphenoxy)benzoic acid | 34043-77-1 | 2 |
| Diatrizoate | 117-96-4 | 3 |
| 2,3,5-Triiodobenzoic acid | 88-82-4 | 3 |
| Acetrizoic acid | 85-36-9 | 3 |
| Metrizoate | 1949-45-7 | 3 |
| 3,4,5-Triiodobenzoic acid | 2338-20-7 | 3 |
| Ioxitalamic acid | 28179-44-4 | 3 |
| Iothalamic acid | 2276-90-6 | 3 |
| 2,4,6-Triiodo-1,3,5-benzenetricarboxylic acid | 79211-41-9 | 3 |
| 2,4,6-Triiodobenzoic acid | 2012-31-9 | 3 |
| enzoic acid, 3-[[[2-hydroxy-1- | 87932-11-4 | 3 |
| (hydroxymethyl)ethyl]amino]carbonyl]-5-[(2- | | |
| hydroxy-1-oxopropyl)amino]-2,4,6-triiodo-,(S)- | | |
| (9CI) | | |
| Ioseric acid | 51876-99-4 | 3 |
| 4-[4-(Acetyloxy)-3-iodophenoxy]-3,5- | 2260-084 | 3 |
| diiodobenzoic acid | | |
| Ioglicic acid | 49755-67-1 | 3 |
| 2,4,6-Triiodo-3-[(1-oxo-3,6,9,12,15- | 16024-67-2 | 3 |
| pentaoxahexadec-1-yl)amino]benzoic acid | | |
| 3-[[(1,1-Dimethylethoxy)carbonyl]amino]-2,4,6- | 2358047-48-8 | 3 |
| triiodobenzoic acid | | |
| 2,3,4,6-Tetraiodobenzoic acid | 71463-71-3 | 4 |
| Tetraform | 2055-97-2 | 4 |
| 2,3,5,6-Tetraiodo-1,4-benzenedicarboxylic acid | 7606-84-0 | 4 |
| N-Acetyl-O-(4-hydroxy-3,5-diiodophenyl)-3,5- | 26041-51-0 | 4 |
| diiodo-L-tyrosine | | |
| D-Tyrosine, N-[(1,1-dimethylethoxy)carbonyl]- | 89624-64-6 | 4 |
| O-(4-hydroxy-3,5-diiodophenyl)-3,5-diiodo- | | |
| (9CI, ACI) | | |
| 2,3,4,5,6-Pentaiodobenzoic acid | 64385-02-0 | 5 |
| Ioxaglic acid | 59017-64-0 | 6 |
| Ioglycamic acid | 2618-25-9 | 6 |
| Iocarmic acid | 10397-75-8 | 6 |

Various amine-containing iodinated compounds, along with their CAS numbers, are listed in the following table.

| Name | CAS | # I atoms |
|---|---|---|
| 4-Iodobenzylamine | 39959-59-6 | 1 |
| 5-Amino-N,N′-bis(2,3-dihydroxypropyl)-2,4,6- | 76801-93-9 | 3 |
| triiodoisophthalamide (iohexol impurity J) | | |
| 3,5-Diiodobenzenamine | 35122-96-4 | 2 |
| 2,4,6-Triiodobenzenamine | 24154-37-8 | 3 |

In some embodiments, an iodinated amino acid is synthesized by reaction of a carboxylic-acid-containing compound and a partially protected amine-based amino acid in which the α-carbon carboxylic acid group and the α-carbon amine group are protected, while the side chain amine group remains unprotected. In this reaction, the carboxylic acid group of the carboxylic-acid-containing iodinated compound is reacted with the unprotected amine group of the partially protected lysine in an amide coupling reaction. The amide coupling is typically performed in the presence of a coupling agent. Coupling agents include carbodiimide coupling agents such as N,N′-dicyclohexylcarbodiimide (DCC), 1-ethyl-3-(3-dimethyl propyl) carbodiimide (EDC), 1,3-diisopropylcarbodiimide (DIC), N-hydroxybenzotriazole (HOBt), 2-(1H-Benzotriazole-1-yl)-1,1,3,3-tetramethylaminium tetrafluoroborate (TBTU), oxalyl chloride, pivaloyl chloride, BOP reagent, and/or another coupling agent.

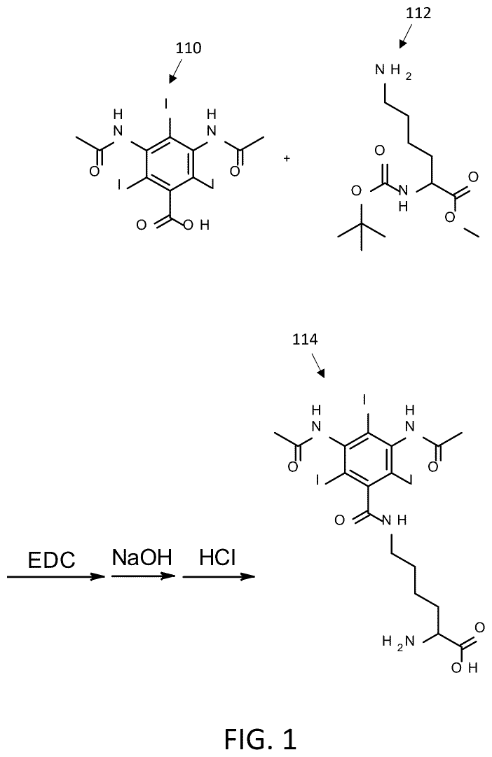

In FIG. 1, the carboxylic acid group of diatrizoic acid 110 is reacted with the side chain amine group of partially protected lysine 112 in the presence of an EDC coupling agent. The α-carbon carboxylic acid group of the lysine is protected by a methyl ester group and the α-carbon amine group is protected by a tert-butoxycarbonyl (1-Boc) group during the amide coupling reaction. Subsequently, the methyl ester is removed by mild hydrolysis with a base such as aqueous NaOH, and deprotection of the t-Boc-protected amine is performed by hydrolysis with an acid such as aqueous HCl or trifluoroacetic acid to produce iodinated amino acid product 114.

In some embodiments, an iodinated amino acid is synthesized by reaction of an amine-containing iodinated compound and a partially protected carboxylic-acid-based amino acid in which the α-carbon carboxylic acid group and the α-carbon amine group are protected, while the side chain carboxylic acid group remains unprotected. In this reaction, the amine group of the amine-containing iodinated compound is reacted with the carboxylic acid group of the side chain in an amide coupling reaction. The amide coupling is typically performed in the presence of a coupling agent. Coupling agents include those listed above, among others. In a particular example (not shown), the amine group of 2,4,6-triiodobenzenamine may be reacted with the side chain carboxylic acid group of partially protected glutamic acid in the presence of an EDC coupling agent. The α-carbon carboxylic acid group of the glutamic acid may be protected by a methyl ester group and the α-carbon amine group may be protected by a t-Boc group during the amide coupling reaction. Subsequently, the methyl ester may be removed by mild hydrolysis with a base such as aqueous NaOH, and deprotection of the t-Boc-protected amine may be performed by hydrolysis with an acid such as aqueous HCl or trifluoroacetic acid, to produce the final iodinated amino acid.

Examples of amine protective groups for amine-based amino acids during amide coupling reactions include tert-butoxycarbonyl (t-Boc) groups, 9-fluorenylmethoxycarbonyl (Fmoc) groups, benzyloxycarbonyl (Cbz) groups, acetamide (Ac) groups, triphenylmethylamine (Tr) groups, trifluoroacetyl (TFA) groups, and 6-nitroveratryloxycarbonyl (Nvoc) groups, among others.

Examples of carboxylic acid protective groups for carboxylic-acid-based amino acids during amide coupling reactions include alkyl esters, particularly methyl ester groups and tert-butyl (1-Bu) ester groups, benzyl ester (β-benzyl) groups, and benzhydryl ester groups, among others.

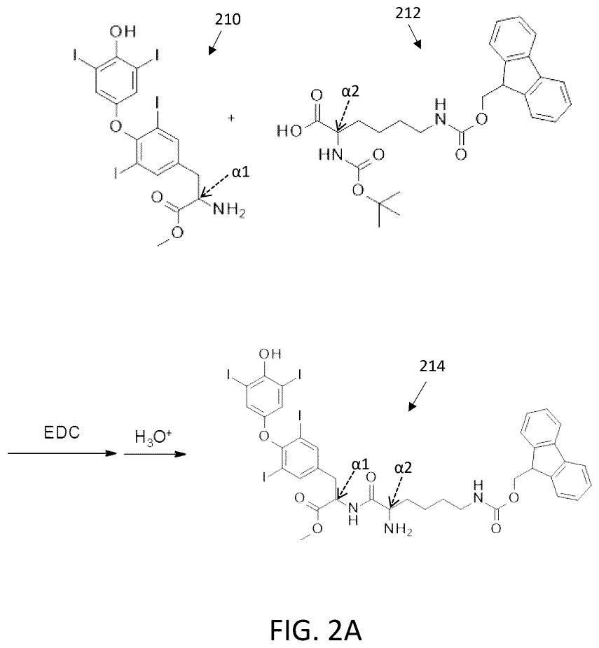

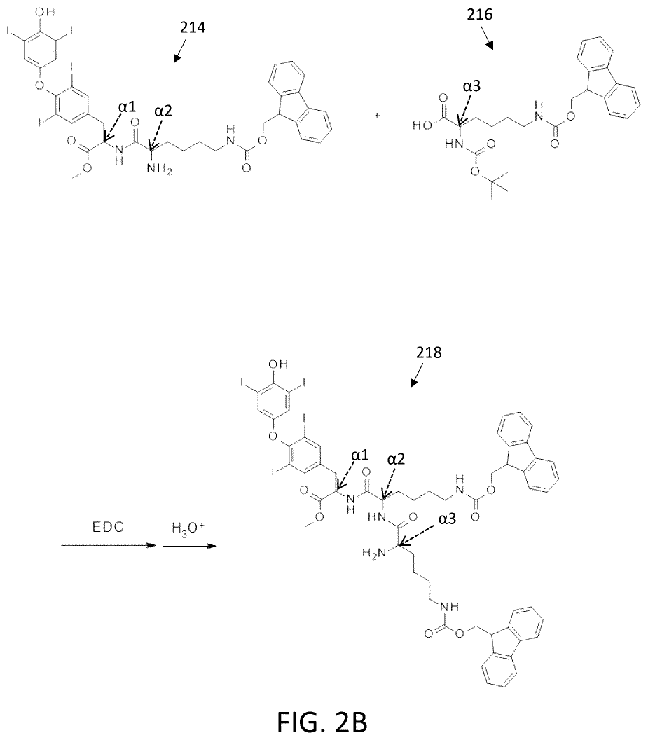

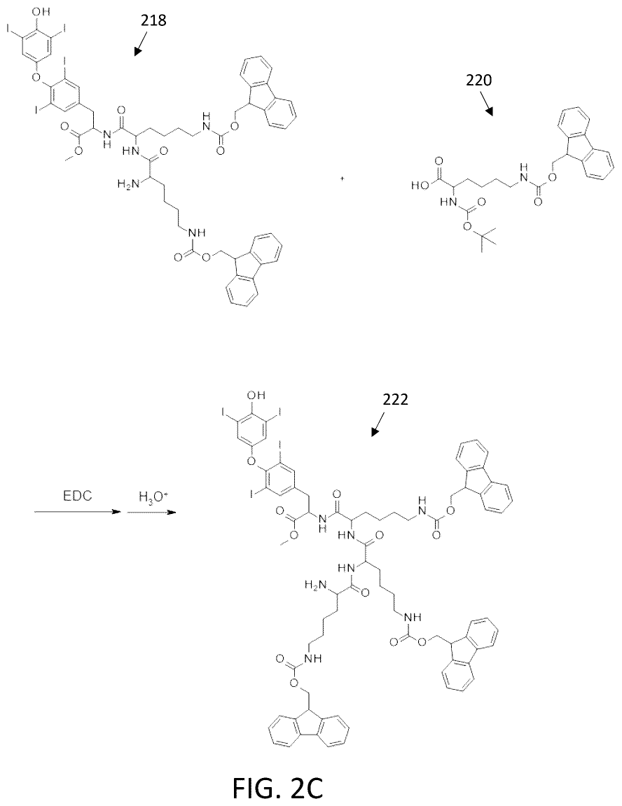

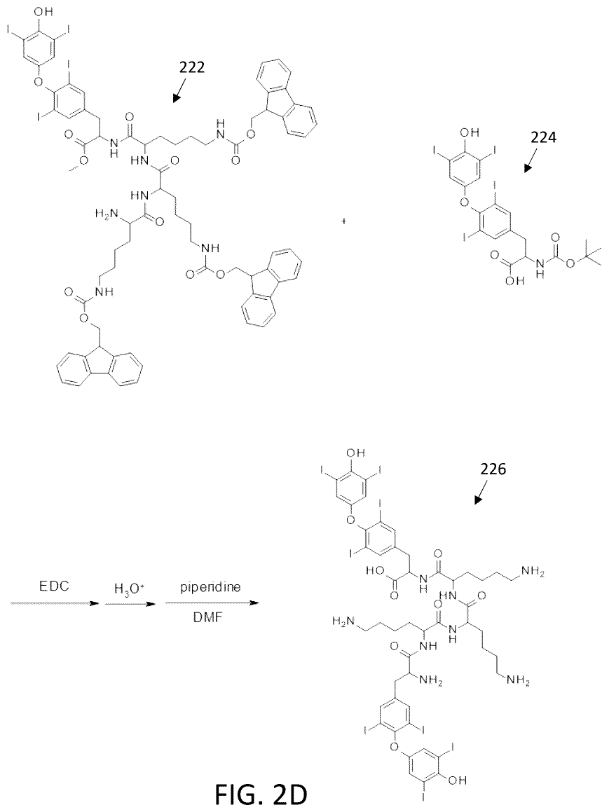

Particular examples of methods of forming radiopaque peptides will now be described. With reference now to FIG. 2A, in a first step an amide coupling reaction is performed between a partially protected first iodinated amino acid, specifically, partially protected thyroxine (Thx) (210), and a partially protected first amine-based amino acid, specifically, partially protected lysine (Lys) (212). The partially protected thyroxine (210) comprises a first α-carbon amino group, a first α-carbon carboxylic acid group and a first iodinated aromatic group, wherein the first α-carbon amino group is unprotected and the first α-carbon carboxylic acid group is protected with a first carboxylic acid protective group, specifically a methyl ester group. The partially protected lysine (212) comprises a second α-carbon amino group, a second α-carbon carboxylic acid group, and a first side chain amino group, wherein the second α-carbon carboxylic acid group is unprotected, the second α-carbon amino group is protected with a first amino protective group, specifically, with a t-Boc group, and the first side chain amino group is protected with a second amino protective group that differs from the first amino protective group, is deprotected using different conditions from those used to deprotect the first amino protective group, and is stable under conditions used to deprotect the first amino group, specifically, an Fmoc group. The first α-carbon (α1) of the partially protected thyroxine (210) and the second α-carbon (α2) of the partially protected lysine (Lys) (212) are designated by dashed arrows in FIG. 2A. The amide linkage is formed by a condensation reaction between the unprotected first α-carbon amino group of the partially protected thyroxine (210) and the unprotected second α-carbon carboxylic acid group of the partially protected lysine (212). The amide coupling reaction of FIG. 2A is performed in the presence of a coupling agent, specifically, EDC. The resulting compound (not separately illustrated) is a partially protected peptide dimer that contains two amino acid residues, specifically, a partially protected Lys-Thx dimer in which the first α-carbon carboxylic acid group is protected by the methyl ester group, the second α-carbon amino group is protected by the t-Boc group, and the first side chain amino group is protected by the Fmoc group.

In a subsequent step, the protection of the second α-carbon amino group is removed while leaving the first side chain amino group and the first α-carbon carboxylic acid group protected. This partial deprotection step is performed by hydrolysis with an acid (H₃O⁺) such as aqueous HCl or trifluoroacetic acid. The resulting compound is a partially protected peptide dimer that contains two amino acid residues, specifically, a partially protected Lys-Thx dimer (214) in which the first α-carbon carboxylic acid group remains protected by the methyl ester group and the first side chain amino group remains protected by the Fmoc group and the second α-carbon amino group (—NH₂ group) is made available for further reaction.

By convention, peptides herein are named starting at the N-terminus of the peptide (i.e., the amine group, —NH₂, end of the peptide) and finishing at the C-terminus of the peptide (i.e., the carboxylic acid, —COOH, end of the peptide).

With reference now to FIG. 2B, in a further step, an amide coupling reaction is performed between the partially protected Lys-Thx dimer (214) product of FIG. 2A and a partially protected further amino acid that comprises a third α-carbon amino group and a third α-carbon carboxylic acid group, wherein the third α-carbon carboxylic acid group is unprotected and the third α-carbon amino group is protected with the first amino protective group. In FIG. 2B, the partially protected further amino acid is an amine-based amino acid that also comprises a second side chain amino group, wherein the second side chain amino group is protected with the second amino protective group. More particularly, the partially protected further amino acid of FIG. 2B is a further partially protected lysine (216) in which the third α-carbon amino group is protected with a t-Boc group and the second side chain amino group is protected with an Fmoc group. The third α-carbon (α3) of the partially protected lysine (216) is designated by a dashed arrow in FIG. 2B. The amide linkage is formed by a condensation reaction between the unprotected second α-carbon amino group of the partially protected dimer (214) and the unprotected third α-carbon carboxylic acid group of the partially protected lysine (216). The amide coupling reaction of FIG. 2B is performed in the presence of a coupling agent, specifically, EDC. The resulting compound (not separately illustrated) is a partially protected peptide trimer that contains three amino acid residues, specifically, a partially protected Lys-Lys-Thx trimer in which the first α-carbon carboxylic acid group is protected by the methyl ester group, the third α-carbon amino group is protected by the t-Boc group, and the first and second side chain amino groups are each protected by the Fmoc group.

In a subsequent step, the protection of the third α-carbon amino group is removed while leaving the first α-carbon carboxylic acid group, the first side chain amino group and the second side chain amino group protected. This partial deprotection step is performed by hydrolysis with an acid (H₃O⁺) such as aqueous HCl or trifluoroacetic acid. The resulting compound is a partially protected peptide trimer that contains three amino acid residues, specifically, a partially protected Lys-Lys-Thx trimer (218) in which the first α-carbon carboxylic acid group remains protected by the methyl ester group, the first side chain amino group remains protected by the Fmoc group, the second side chain amino group remains protected by the Fmoc group, and the third α-carbon amino group (—NH₂ group) is made available for further reaction.

As will become apparent from disclosure to follow, although the further amino acid of FIG. 2B is an amine-based amino acid, a variety of amino acids can be used in its place, including iodinated amino acids and carboxylic-acid-based amino acids.

With reference now to FIG. 2C, in a further step, an amide coupling reaction is performed between the partially protected Lys-Lys-Thx trimer (218) product of FIG. 2B and a partially protected further amino acid that comprises a fourth α-carbon amino group and a fourth α-carbon carboxylic acid group, wherein the fourth α-carbon carboxylic acid group is unprotected and the fourth α-carbon amino group is protected with the first amino protective group. In FIG. 2C, the partially protected further amino acid also comprises a third side chain amino group, wherein the third side chain amino group is protected with the second amino protective group. More particularly, the partially protected further amino acid of FIG. 2C is another partially protected lysine (220) in which the fourth α-carbon amino group is protected with a t-Boc group and the third side chain amino group is protected with an Fmoc group. The amide linkage is formed by a condensation reaction between the unprotected third α-carbon amino group of the partially protected trimer (218) and the unprotected fourth α-carbon carboxylic acid group of the partially protected lysine (220). The amide coupling reaction of FIG. 2C is performed in the presence of a coupling agent, specifically, EDC. The resulting compound (not separately illustrated) is a partially protected peptide tetramer that contains four amino acid residues, specifically, a partially protected Lys-Lys-Lys-Thx tetramer in which the first α-carbon carboxylic acid group is protected by the methyl ester group, the fourth α-carbon amino group is protected by the t-Boc group, and the first, second and third side chain amino groups are each protected by an Fmoc group.

In a subsequent step, the protection of the fourth α-carbon amino group is removed while leaving the first α-carbon carboxylic acid group, the first side chain amino group, the second side chain amino group, and the third side chain amino group protected. This partial deprotection step is performed by hydrolysis with an acid (H₃O⁺) such as aqueous HCl or trifluoroacetic acid. The resulting compound is a partially protected peptide tetramer that contains four amino acid residues, specifically, a partially protected Lys-Lys-Lys-Thx tetramer (222) in which the first α-carbon carboxylic acid group remains protected by the methyl ester group, the first side chain amino group remains protected by the Fmoc group, the second side chain amino group remains protected by the Fmoc group, the third side chain amino group remains protected by the Fmoc group, and the fourth α-carbon amino group is made available for further reaction.

Although the further amino acid of FIG. 2C is an amine-based amino acid, a variety of amino acids can be used in its place, including iodinated amino acids and carboxylic-acid-based amino acids.

With reference now to FIG. 2D, in a further step, an amide coupling reaction is performed between the partially protected Lys-Lys-Lys-Thx tetramer (222) product of FIG. 2C and a partially protected further amino acid that comprises a fifth α-carbon amino group and a fifth α-carbon carboxylic acid group, wherein the fifth α-carbon carboxylic acid group is unprotected and the fifth α-carbon amino group is protected with the first amino protective group. In FIG. 2D, the partially protected further amino acid also comprises an iodinated aromatic group. More particularly, the partially protected further amino acid of FIG. 2D is partially protected thyroxine (224) in which the fifth α-carbon carboxylic acid group is unprotected and the fifth α-carbon amino group is protected with a t-Boc group. The amide linkage is formed by a condensation reaction between the unprotected fourth α-carbon amino group of the partially protected tetramer (222) and the unprotected fifth α-carbon carboxylic acid group of the partially protected thyroxine (224). The amide coupling reaction of FIG. 2D is performed in the presence of a coupling agent, specifically, EDC. The resulting compound (not separately illustrated) is a partially protected peptide pentamer that contains five amino acid residues, specifically, a partially protected Thx-Lys-Lys-Lys-Thx pentamer in which the first α-carbon carboxylic acid group is protected by the methyl ester group, the fifth α-carbon amino group is protected by the t-Boc group, and the first, second and third side chain amino groups are each protected by an Fmoc group.

Although the further amino acid of FIG. 2D is an iodinated amino acid, a variety of amino acids can be used in its place, including amine-based amino acids and carboxylic-acid-based amino acids.

At this point the target radiopaque peptide has been achieved, and all protective groups are removed to yield the final radiopaque peptide, specifically, a Thx-Lys-Lys-Lys-Thx pentamer (226). In the particular embodiment of FIG. 2D, the t-Boc protection of the fifth α-carbon amino group is removed by hydrolysis with an acid (H₃O⁺) such as aqueous HCl or trifluoroacetic acid, the methyl ester protection of the first α-carbon carboxylic acid group is removed by any suitable technique, for example, by saponification with NaOH, and the Fmoc protection of the first, second and third side chain amino groups are removed by any suitable technique, for example by treatment with a primary (e.g., cyclohexylamine, ethanolamine) or secondary (e.g., piperidine, piperazine) amine. In the particular embodiment shown, the Fmoc protection is removed by treatment with piperidine in tetrahydrofuran (THF).

The final radiopaque peptides produced herein are shown as free bases. Formation of a salt may be obtained via acid treatment, for example, HCl, to form the hydrochloride salt, or acetic acid, to form the acetate salt. The salt will improve water solubility of the crosslinker, which is important for in-vivo applications.

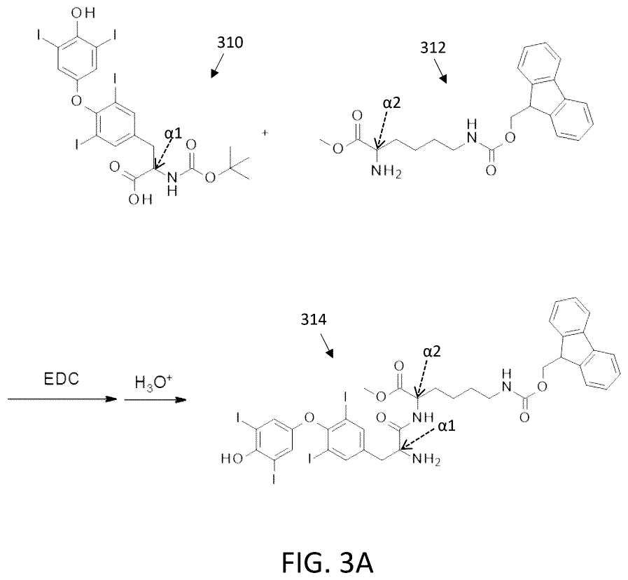

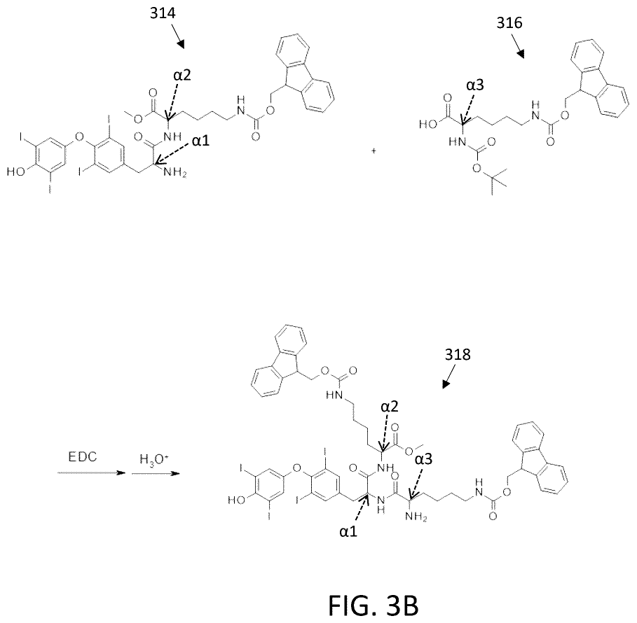

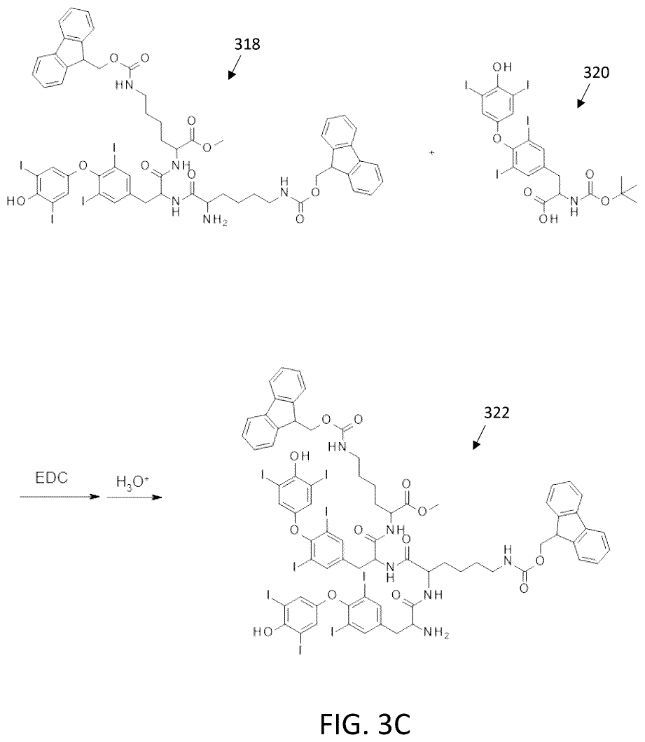

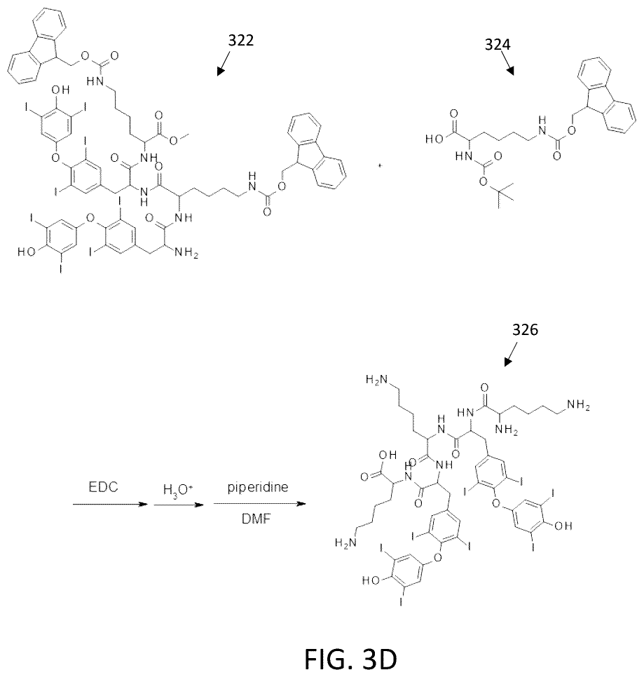

Another example of a method of forming a radiopaque peptide will now be described in conjunction with FIGS. 3A-3D. In a first step shown in FIG. 3A, an amide coupling reaction is performed between a partially protected first iodinated amino acid, specifically, partially protected thyroxine (310), and a partially protected first amine-based amino acid, specifically, partially protected lysine (312). The partially protected thyroxine (310) comprises a first α-carbon amino group, a first α-carbon carboxylic acid group and a first iodinated aromatic group, wherein the first α-carbon carboxylic acid group is unprotected and the first α-carbon amino group is protected with a first amino protective group, specifically, with a t-Boc group. The partially protected lysine (312) comprises a second α-carbon amino group, a second α-carbon carboxylic acid group, and a first side chain amino group, wherein the second α-carbon carboxylic acid group is protected with a first carboxylic acid protective group, specifically a methyl ester group, the second α-carbon amino group is unprotected, and the first side chain amino group is protected with a second amino protective group that differs from the first amino protective group, is deprotected using different conditions from those used to deprotect the first amino protective group, and is stable under conditions used to deprotect the first amino group, specifically, with an Fmoc group. The second α-carbon (α2) of the partially protected lysine (312) and the first α-carbon (α1) of the partially protected thyroxine (310) are designated by dashed arrows in FIG. 3A. The amide linkage is formed by a condensation reaction between the unprotected first α-carbon carboxylic acid group of the partially protected thyroxine (310) and the unprotected second α-carbon amino of the partially protected lysine (312). The amide coupling reaction of FIG. 3A is performed in the presence of a coupling agent, specifically, EDC. The resulting compound (not separately illustrated) is a partially protected peptide dimer that contains two amino acid residues, specifically, a partially protected Thx-Lys dimer in which the first α-carbon amino group is protected by the t-Boc group, the second α-carbon carboxylic acid group is protected by the methyl ester group, and the first side chain amino group is protected by the Fmoc group.

In a subsequent step, the protection of the first α-carbon amino group is removed while leaving the second α-carbon carboxylic acid group and the first side chain amino group protected. This partial deprotection step is performed by hydrolysis with an acid (H₃O⁺) such as aqueous HCl or trifluoroacetic acid. The resulting compound is a partially protected peptide dimer that contains two amino acid residues, specifically, a partially protected Thx-Lys dimer (314) in which the first α-carbon amino group (—NH₂ group) is made available for further reaction, the second α-carbon carboxylic acid group remains protected by the methyl ester group, and the first side chain amino group remains protected by the Fmoc group.

With reference now to FIG. 3B, in a further step, an amide coupling reaction is performed between the partially protected Thx-Lys dimer (314) product of FIG. 3A and a partially protected further amino acid that comprises a third α-carbon amino group and a third α-carbon carboxylic acid group, wherein the third α-carbon amino group is protected with the first amino protective group (t-Boc group) and the third α-carbon carboxylic acid group is unprotected. In FIG. 3B, the partially protected further amino acid also comprises a second side chain amino group, wherein the second side chain amino group is protected with the second amino protective group (Fmoc group). More particularly, the partially protected further amino acid of FIG. 3B is a further partially protected lysine (316) in which the third α-carbon amino group is protected with a t-Boc group and the second side chain amino group is protected with an Fmoc group. The third α-carbon (α3) of the partially protected lysine (316) is designated by a dashed arrow in FIG. 3B. The amide linkage is formed by a condensation reaction between the deprotected first α-carbon amino group of the partially protected dimer (314) and the unprotected third α-carbon carboxylic acid group of the partially protected lysine (316). The amide coupling reaction of FIG. 3B is performed in the presence of EDC as a coupling agent. The resulting compound (not separately illustrated) is a partially protected peptide trimer that contains three amino acid residues, specifically, a partially protected Lys-Thx-Lys trimer in which the second α-carbon carboxylic acid group is protected by the methyl ester group, the third α-carbon amino group is protected by the t-Boc group, and the first and second side chain amino groups are each protected by the Fmoc group.

In a subsequent step, the protection of the third α-carbon amino group is removed while leaving the second α-carbon carboxylic acid group, the first side chain amino group and the second side chain amino group protected. This partial deprotection step is performed by hydrolysis with an acid (H₃O⁺) such as aqueous HCl or trifluoroacetic acid. The resulting compound is a partially protected peptide trimer that contains three amino acid residues, specifically, a partially protected Lys-Thx-Lys trimer (318) in which the second α-carbon carboxylic acid group remains protected by the methyl ester group, the first side chain amino group remains protected by the Fmoc group, the second side chain amino group remains protected by the Fmoc group, and the third α-carbon amino group (—NH₂ group) is made available for further reaction.

Although the further amino acid of FIG. 3B is an amine-based amino acid, a variety of amino acids can be used in its place, including iodinated amino acids and carboxylic-acid-based amino acids.

With reference now to FIG. 3C, in a further step, an amide coupling reaction is performed between the partially protected Lys-Thx-Lys trimer (318) product of FIG. 3B and a partially protected further amino acid that comprises a fourth α-carbon amino group and a fourth α-carbon carboxylic acid group, wherein the fourth α-carbon carboxylic acid group is unprotected and the fourth α-carbon amino group is protected with the first amino protective group. In FIG. 3C, the partially protected further amino acid also comprises an iodinated aromatic group. More particularly, the partially protected further amino acid of FIG. 3C is partially protected thyroxine (320) in which the fourth α-carbon amino group is protected with a t-Boc group. The amide linkage is formed by a condensation reaction between the unprotected third α-carbon amino group of the partially protected trimer (318) and the unprotected fourth α-carbon carboxylic acid group of the partially protected thyroxine (320). The amide coupling reaction of FIG. 3C is performed in the presence of EDC as a coupling agent. The resulting compound (not separately illustrated) is a partially protected peptide tetramer that contains four amino acid residues, specifically, a partially protected Thx-Lys-Thx-Lys tetramer in which the second α-carbon carboxylic acid group is protected by the methyl ester group, the fourth α-carbon amino group is protected by the t-Boc group, and the first and second chain amino groups are each protected by an Fmoc group.

In a subsequent step, the protection of the fourth α-carbon amino group is removed while leaving the second α-carbon carboxylic acid group, the first side chain amino group and the second side chain amino group protected. This partial deprotection step is performed by hydrolysis with an acid (H₃O⁺) such as aqueous HCl or trifluoroacetic acid. The resulting compound is a partially protected peptide tetramer that contains four amino acid residues, specifically, a partially protected Thx-Lys-Thx-Lys tetramer (322) in which the second α-carbon carboxylic acid group remains protected by the methyl ester group, the first side chain amino group remains protected by the Fmoc group, the second side chain amino group remains protected by the Fmoc group, and the fourth α-carbon amino group (—NH₂ group) is made available for further reaction.

Although the further amino acid of FIG. 3C is an iodinated amino acid, a variety of amino acids can be used in its place, including amine-based amino acids and carboxylic-acid-based amino acids.

With reference now to FIG. 3D, in a further step, an amide coupling reaction is performed between the partially protected Thx-Lys-Thx-Lys tetramer (322) product of FIG. 3C and a partially protected further amino acid that comprises a fifth α-carbon amino group and a fifth α-carbon carboxylic acid group, wherein the fifth α-carbon carboxylic acid group is unprotected and the fifth α-carbon amino group is protected with the first amino protective group. In FIG. 3D, the partially protected further amino acid also comprises a third side chain amino group, wherein the third side chain amino group is protected with the second amino protective group (Fmoc group). More particularly, the partially protected further amino acid of FIG. 3D is a partially protected lysine (Lys) (316) in which the fifth α-carbon carboxylic acid group is unprotected, the fifth α-carbon amino group is protected with a t-Boc group and the third side chain amino group is protected with an Fmoc group. The amide linkage is formed by a condensation reaction between the unprotected fourth α-carbon amino group of the partially protected tetramer (322) and the unprotected fifth α-carbon carboxylic acid group of the partially protected lysine (324). The amide coupling reaction of FIG. 3D is performed in the presence of EDC as a coupling agent. The resulting compound (not separately illustrated) is a partially protected peptide pentamer that contains five amino acid residues, specifically, a partially protected Lys-Thx-Lys-Thx-Lys pentamer in which the second α-carbon carboxylic acid group is protected by the methyl ester group, the fifth α-carbon amino group is protected by the t-Boc group, and the first, second and third side chain amino groups are each protected by an Fmoc group.

At this point the target radiopaque peptide has been achieved, and all protective groups are removed to yield the final radiopaque peptide, specifically, a Lys-Thx-Lys-Thx-Lys pentamer (326). In the particular embodiment of FIG. 3D, the t-Boc protection of the fifth α-carbon amino group is removed by hydrolysis with an acid (H₃O⁺) such as aqueous HCl or trifluoroacetic acid, the methyl ester protection of the second α-carbon carboxylic acid group is removed by any suitable technique, for example, by saponification with NaOH, and the Fmoc protection of the first, second and third side chain amino groups are removed by any suitable technique, for example, by treatment with primary (e.g., cyclohexylamine, ethanolamine) or secondary (e.g., piperidine, piperazine) amines. In the particular embodiment shown, the Fmoc protection is removed by treatment with piperidine in tetrahydrofuran (THF).

Although the further amino acid of FIG. 3D is an amine-based amino acid, a variety of amino acids can be used in its place, including iodinated amino acids and carboxylic-acid-based amino acids.

Moreover, although the final Lys-Thx-Lys-Thx-Lys pentamer (326) produced is FIG. 3D is shown a free base, formation of a salt may be obtained via acid treatment, for example, HCl, to form the hydrochloride salt, or acetic acid, to form the acetate salt.

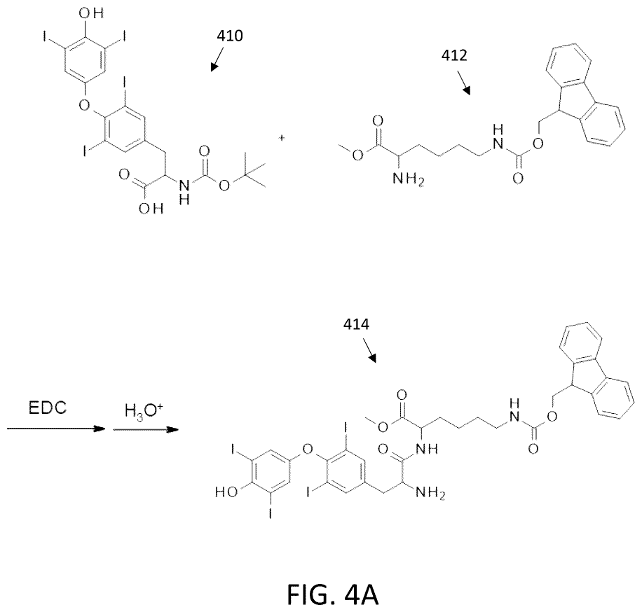

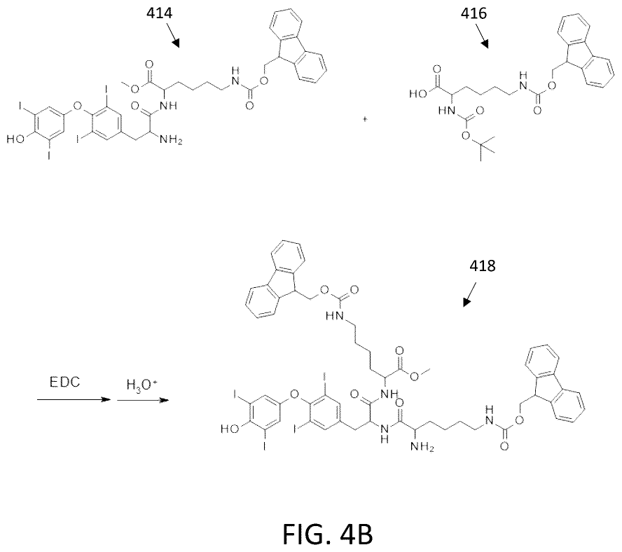

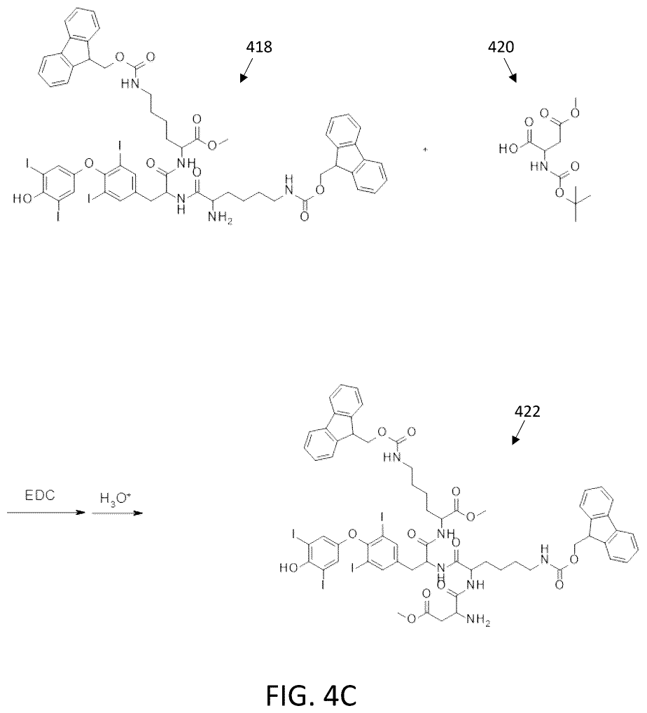

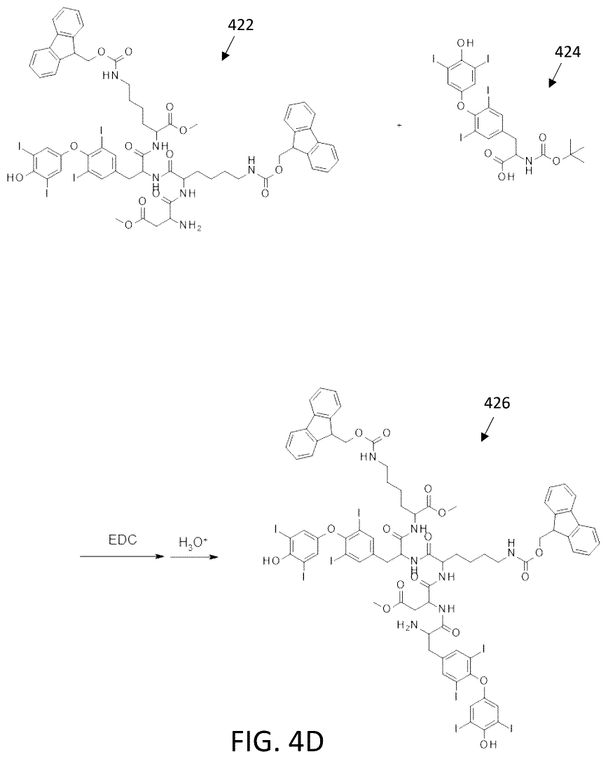

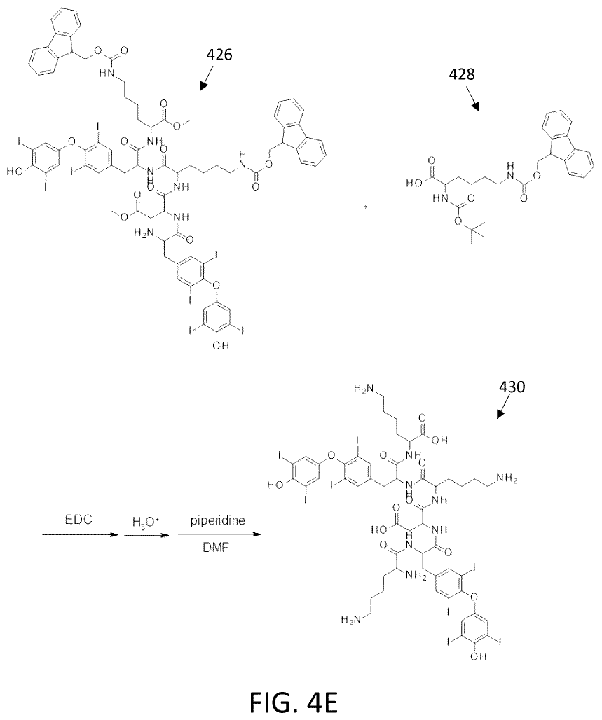

Another example of a method of forming a radiopaque peptide will now be described in conjunction with FIGS. 4A-4E. In a first step shown in FIG. 4A, an amide coupling reaction is performed between a partially protected first iodinated amino acid, specifically, partially protected thyroxine (Thx) (410) and a partially protected first amine-based amino acid, specifically, partially protected lysine (Lys) (412). The partially protected thyroxine (Thx) (410) comprises a first α-carbon amino group, a first α-carbon carboxylic acid group and a first iodinated aromatic group, wherein the first α-carbon carboxylic acid group is unprotected and the first α-carbon amino group is protected with a first amino protective group, specifically, with a t-Boc group. The partially protected lysine (Lys) (412) comprises a second α-carbon amino group, a second α-carbon carboxylic acid group, and a first side chain amino group, wherein the second α-carbon carboxylic acid group is protected with a first carboxylic acid protective group, specifically a methyl ester group, the second α-carbon amino group is unprotected, and the first side chain amino group is protected with a second amino protective group that differs from the first amino protective group, specifically, with an Fmoc group. The amide linkage is formed by a condensation reaction between the unprotected first α-carbon carboxylic acid group of the partially protected thyroxine (410) and the unprotected second α-carbon amino of the partially protected lysine (412). The amide coupling reaction of FIG. 4A is performed in the presence of a coupling agent, specifically, EDC. The resulting compound (not separately illustrated) is a partially protected peptide dimer that contains two amino acid residues, specifically, a partially protected Thx-Lys dimer in which the first α-carbon amino group is protected by the t-Boc group, the second α-carbon carboxylic acid group is protected by the methyl ester group, and the first side chain amino group is protected by the Fmoc group.

In a subsequent step, the t-Boc protection of the first α-carbon amino group is removed while leaving the second α-carbon carboxylic acid group and the first side chain amino group protected. This partial deprotection step is performed by hydrolysis with an acid (H₃O⁺) such as aqueous HCl or trifluoroacetic acid. The resulting compound is a partially protected peptide dimer that contains two amino acid residues, specifically, a partially protected Thx-Lys dimer (414) in which the first α-carbon amino group (—NH₂ group) is made available for further reaction, the second α-carbon carboxylic acid group remains protected by the methyl ester group, and the first side chain amino group remains protected by the Fmoc group.

With reference now to FIG. 4B, in a further step, an amide coupling reaction is performed between the partially protected Thx-Lys dimer (414) product of FIG. 4A and a partially protected further amino acid that comprises a third α-carbon amino group and a third α-carbon carboxylic acid group, wherein the third α-carbon amino group is protected with the first amino protective group (t-Boc group) and the third α-carbon carboxylic acid group is unprotected. In FIG. 4B, the partially protected further amino acid also comprises a second side chain amino group, wherein the second side chain amino group is protected with the second amino protective group (Fmoc group). More particularly, the partially protected further amino acid of FIG. 4B is a partially protected lysine (Lys) (416) in which the third α-carbon amino group is protected with a t-Boc group and the second side chain amino group is protected with an Fmoc group. The amide linkage is formed by a condensation reaction between the deprotected first α-carbon amino group of the partially protected dimer (414) and the unprotected third α-carbon carboxylic acid group of the partially protected lysine (416). The amide coupling reaction of FIG. 4B is performed in the presence of EDC as a coupling agent. The resulting compound (not separately illustrated) is a partially protected peptide trimer that contains three amino acid residues, specifically, a partially protected Lys-Thx-Lys trimer in which the second α-carbon carboxylic acid group is protected by the methyl ester group, the third α-carbon amino group is protected by the t-Boc group, and the first and second side chain amino groups are each protected by the Fmoc group.

In a subsequent step, the t-Boc protection of the third α-carbon amino group is removed while leaving the second α-carbon carboxylic acid group, the first side chain amino group and the second side chain amino group protected. This partial deprotection step is performed by hydrolysis with an acid (H₃O⁺) such as aqueous HCl or trifluoroacetic acid. The resulting compound is a partially protected peptide trimer that contains three amino acid residues, specifically, a partially protected Lys-Thx-Lys trimer (418) in which the second α-carbon carboxylic acid group remains protected by the methyl ester group, the first side chain amino group remains protected by the Fmoc group, the second side chain amino group remains protected by the Fmoc group, and the third α-carbon amino group (—NH₂ group) is made available for further reaction.

Although the further amino acid of FIG. 4B is an amine-based amino acid, a variety of amino acids can be used in its place, including iodinated amino acids and carboxylic-acid-based amino acids.

With reference now to FIG. 4C, in a further step, an amide coupling reaction is performed between the partially protected Lys-Thx-Lys trimer (418) product of FIG. 4B and a partially protected further amino acid that comprises a fourth α-carbon amino group and a fourth α-carbon carboxylic acid group, wherein the fourth α-carbon carboxylic acid group is unprotected and the fourth α-carbon amino group is protected with the first amino protective group. In FIG. 4C, the partially protected further amino acid is a carboxylic-acid-based amino acid that also comprises a first side chain carboxylic acid group, wherein the first side chain carboxylic acid group is protected with a suitable protective group, such as an alkyl ester group. More particularly, the partially protected further amino acid of FIG. 4C is partially protected aspartic acid (Asp) (420) in which the fourth α-carbon amino group is protected with a t-Boc group and the first side chain carboxylic acid group is protected with a methyl ester group. The amide linkage is formed by a condensation reaction between the unprotected third α-carbon amino group of the partially protected trimer (418) and the unprotected fourth α-carbon carboxylic acid group of the partially protected aspartic acid (420). The amide coupling reaction of FIG. 4C is performed in the presence of EDC as a coupling agent. The resulting compound (not separately illustrated) is a partially protected peptide tetramer that contains four amino acid residues, specifically, a partially protected Asp-Lys-Thx-Lys tetramer in which the second α-carbon carboxylic acid group is protected by the methyl ester group, the fourth α-carbon amino group is protected by the t-Boc group, the first side chain carboxylic acid group is protected with a methyl ester group, and the first and second chain amino groups are each protected by an Fmoc group.

In a subsequent step, the t-Boc protection of the fourth α-carbon amino group is removed while leaving the second α-carbon carboxylic acid group, the first side chain carboxylic acid group, the first side chain amino group, and the second side chain amino group protected. This partial deprotection step is performed by hydrolysis with an acid (H₃O⁺) such as aqueous HCl or trifluoroacetic acid. The resulting compound is a partially protected peptide tetramer that contains four amino acid residues, specifically, a partially protected Asp-Lys-Thx-Lys tetramer (422) in which the second α-carbon carboxylic acid group remains protected by the methyl ester group, the first side chain carboxylic acid group is protected with the methyl ester group, the first side chain amino group remains protected by the Fmoc group, the second side chain amino group remains protected by the Fmoc group, and the fourth α-carbon amino group (—NH₂ group) is made available for further reaction.

Although the further amino acid of FIG. 4C is a carboxylic-acid-based amino acid, a variety of amino acids can be used in its place, including iodinated amino acids and amine-based amino acids.

With reference now to FIG. 4D, in a further step, an amide coupling reaction is performed between the partially protected Asp-Lys-Thx-Lys tetramer (422) product of FIG. 4C and a partially protected further amino acid that comprises a fifth α-carbon amino group and a fifth α-carbon carboxylic acid group, wherein the fifth α-carbon carboxylic acid group is unprotected and the fifth α-carbon amino group is protected with the first amino protective group. In FIG. 4D, the partially protected further amino acid also comprises an iodinated aromatic group. More particularly, the partially protected further amino acid of FIG. 4D is partially protected thyroxine (424) in which the fifth α-carbon amino group is protected with a t-Boc group. The amide linkage is formed by a condensation reaction between the unprotected fourth α-carbon amino group of the partially protected tetramer (422) and the unprotected fifth α-carbon carboxylic acid group of the partially protected thyroxine (424). The amide coupling reaction of FIG. 4D is performed in the presence of EDC as a coupling agent. The resulting compound (not separately illustrated) is a partially protected peptide pentamer that contains five amino acid residues, specifically, a partially protected Thx-Asp-Lys-Thx-Lys pentamer in which the second α-carbon carboxylic acid group is protected by the methyl ester group, the first side chain carboxylic acid group is protected with the methyl ester group, the fifth α-carbon amino group is protected by the t-Boc group, and the first and second side chain amino groups are each protected by an Fmoc group.

In a subsequent step, the protection of the fifth α-carbon amino group is removed while leaving the second α-carbon carboxylic acid group, the first side chain carboxylic acid group, the first side chain amino group and the second side chain amino group protected. This partial deprotection step is performed by hydrolysis with an acid (H₃O⁺) such as aqueous HCl or trifluoroacetic acid. The resulting compound is a partially protected peptide pentamer that contains five amino acid residues, specifically, a partially protected Thx-Asp-Lys-Thx-Lys pentamer (426) in which the second α-carbon carboxylic acid group is protected by the methyl ester group, the first side chain carboxylic acid group is protected with the methyl ester group, and the first and second side chain amino groups are each protected by an Fmoc group.

Although the further amino acid of FIG. 4D is an iodinated amino acid, a variety of amino acids can be used in its place, including amine-based amino acids and carboxylic-acid-based amino acids.

With reference now to FIG. 4E, in a further step, an amide coupling reaction is performed between the partially protected partially protected Thx-Asp-Lys-Thx-Lys pentamer (426) product of FIG. 4D and a partially protected further amino acid that comprises a sixth α-carbon amino group and a sixth α-carbon carboxylic acid group, wherein the sixth α-carbon carboxylic acid group is unprotected and the sixth α-carbon amino group is protected with the first amino protective group. In FIG. 4E, the partially protected further amino acid also comprises a third side chain amino group, wherein the third side chain amino group is protected with the second amino protective group (Fmoc group). More particularly, the partially protected further amino acid of FIG. 4E is a partially protected lysine (428) in which the sixth α-carbon amino group is protected with a t-Boc group and the third side chain amino group is protected with an Fmoc group. The amide linkage is formed by a condensation reaction between the unprotected fifth α-carbon amino group of the partially protected pentamer (426) and the unprotected sixth α-carbon carboxylic acid group of the partially protected lysine (428). The amide coupling reaction of FIG. 4E is performed in the presence of EDC as a coupling agent. The resulting compound (not separately illustrated) is a partially protected peptide hexamer that contains six amino acid residues, specifically, a partially protected Lys-Thx-Asp-Lys-Thx-Lys hexamer in which the second α-carbon carboxylic acid group is protected by the methyl ester group, the first side chain carboxylic acid group is protected with the methyl ester group, the sixth α-carbon amino group is protected by the t-Boc group, and the first, second and third side chain amino groups are each protected by an Fmoc group.

At this point the target radiopaque peptide has been achieved, and all protective groups are removed to yield the final radiopaque peptide, specifically, a Lys-Thx-Asp-Lys-Thx-Lys hexamer (430). In the particular embodiment of FIG. 4E, the t-Boc protection of the sixth α-carbon amino group is removed by hydrolysis with an acid (H₃O⁺) such as aqueous HCl or trifluoroacetic acid, the methyl ester protection of the second α-carbon carboxylic acid group and the first side chain carboxylic acid group is removed by any suitable technique, for example, by saponification with NaOH, and the Fmoc protection of the first, second and third side chain amino groups are removed by any suitable technique, for example, by treatment with a primary (e.g., cyclohexylamine, ethanolamine) or secondary (e.g., piperidine, piperazine) amine. In the particular embodiment shown, the Fmoc protection is removed by treatment with piperidine in tetrahydrofuran (THF).

Although the further amino acid of FIG. 4E is an amine-based amino acid, a variety of amino acids can be used in its place, including iodinated amino acids and carboxylic-acid-based amino acids.

Moreover, although the final Lys-Thx-Asp-Lys-Thx-Lys hexamer (430) produced is FIG. 4E is shown a free base, formation of a salt may be obtained via acid treatment, for example, HCl, to form the hydrochloride salt, or acetic acid, to form the acetate salt.

In other aspects of the present disclosure, crosslinked networks are formed by reacting (a) a radiopaque peptide compound formed in accordance with the present disclosure, which comprises two or more primary-amine-containing side groups, and (b) a reactive polymer that comprises moieties that are reactive with the primary amine groups of the radiopaque peptide.

In some embodiments the crosslinked networks are hydrogels. As used herein, a “hydrogel” (also referred to as a “crosslinked hydrogel”) is a crosslinked polymer that can absorb water but does not dissolve when placed in water.

The crosslinked hydrogels of the present disclosure may be formed in vivo (e.g., using a delivery device like that described below), or such crosslinked hydrogels may be formed ex vivo and subsequently administered to a subject. The crosslinked hydrogels of the present disclosure may be used in a variety of biomedical applications, including implants, medical devices, and pharmaceutical compositions.

In various embodiments, the crosslinked hydrogel is visible under fluoroscopy. The crosslinked hydrogel may have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values).

Reactive polymers for use in the present disclosure include reactive multi-arm polymers that comprise a plurality of polymer arms linked to a core region, at least a portion of the arms comprising a hydrophilic polymer segment. One end of the hydrophilic polymer segment is covalently linked to the core region and an opposite end of the hydrophilic polymer segment is covalently linked to a reactive moiety.

In certain embodiments, at least a portion of the polymer arms comprise a hydrophilic polymer segment that has first and second ends, the first end of the hydrophilic polymer segment covalently linked to the core region, a cyclic anhydride residue having first and second ends, the first end of the cyclic anhydride residue covalently linked to the second end of the hydrophilic polymer segment, and a reactive moiety that is covalently linked to the second end of the cyclic anhydride residue.

Reactive polymers in accordance with the present disclosure include polymers having from 3 to 100 arms, for example ranging anywhere from 3 to 4 to 5 to 6 to 7 to 8 to 10 to 12 to 15 to 20 to 25 to 50 to 75 to 100 arms (in other words, having a number of arms ranging between any two of the preceding values).

Reactive moieties include moieties that comprise electrophilic groups.

Electrophilic groups may be selected, for example, from cyclic imide ester groups, such as succinimide ester groups,

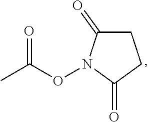

maleimide ester groups, glutarimide ester groups, diglycolimide ester groups, phthalimide ester groups, and bicyclo[2.2.1]hept-5-ene-2,3-dicarboxylic acid imide ester groups,

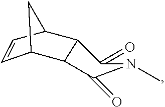

imidazole ester groups, imidazole carboxylate groups and benzotriazole ester groups, among other possibilities.

The electrophilic groups may be linked to the hydrophilic polymer segment through any suitable linking moiety, which may be selected, for example, from a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, or a linking moiety that comprises a combination of two or more of the foregoing groups, among others. In certain embodiments, the linking moiety comprises a hydrolysable ester group.

Hydrophilic polymer segments for the polymer arms can be selected from a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, and polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the multi-arm polymers of the present disclosure typically contain between 2 and 1000 monomer units or more, for example, ranging anywhere from 2 to 3 to 4 to 6 to 8 to 10 to 15 to 20 to 25 to 50 to 100 to 250 to 500 to 1000 monomer units (in other words range between any two of the preceding values).

In certain embodiments, the core region comprises a residue of a polyol comprising three or more hydroxyl groups, which is used to form the polymer arms. In certain beneficial embodiments, the core region comprises a residue of a polyol that contains from 3 to 100 hydroxyl groups, for example, ranging 2 to 3 to 4 to 5 to 6 to 8 to 10 to 15 to 20 to 25 to 50 to 75 to 100 hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, nonamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl) ethane, and 2,6-bis(hydroxyalkyl) cresols, among others.

Illustrative polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 3 to 100 monomer units in length.

In other embodiments, the core region comprises a silsesquioxane, which is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, the organic groups comprise polymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T6, T8, T10, and T12 cage-like silicon-oxygen cores, respectively (where T=the number of tetrahedral Si vertices). In all cases each Si atom is bonded to three O atoms, which in turn connect to other Si atoms. Silsesquioxanes include compounds of the chemical formula [RSiO_{3/2}]ₙ, where n is an integer of at least 6, commonly 6, 8, 10 or 12 (thereby having T6, T8, T₁₀ or T₁₂ cage-like silicon-oxygen core, respectively), and where R may be selected from an array of organic functional groups such as alkyl groups, aryl groups, alkoxyl groups, and polymeric arms, among others. The T₈ cage-like silicon-oxygen cores are widely studied and have the formula [RSiO_{3/2}]₈, or equivalently R₈Si₈O₁₂. Such a structure is shown here:

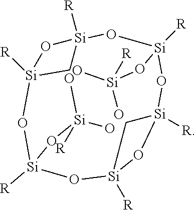

In the present disclosure, the R groups comprise the polymer arms described herein.

Reactive multi-arm polymers in accordance with the present disclosure can be formed from hydroxy-terminated precursor multi-arm polymers having arms that comprise one or more hydroxyl end groups. In some of these embodiments, the hydroxy-terminated precursor multi-arm hydrophilic polymer may be reacted with a cyclic anhydride to form an acid-end-capped precursor polymer. For example, terminal hydroxyl groups of the hydrophilic segments may be reacted with a cyclic anhydride (e.g., a glutaric anhydride compound, a succinic anhydride compound, a malonic anhydride compound, an adipic anhydride compound, a diglycolic anhydride compound, etc.) to form an acid-end-capped segment such as a glutaric-acid-end-capped segment, a succinic-acid-end-capped segment, a malonic-acid-end-capped segment, an adipic-acid-end-capped segment, a diglycolic-acid-end-capped segment, and so forth.

The preceding cyclic anhydrides, among others, may be reacted with a hydroxy-terminated precursor multi-arm hydrophilic polymer under basic conditions to form a carboxylic-acid-terminated precursor polymer comprising a carboxylic acid end group that is linked to a hydrophilic polymer segment through a hydrolysable ester group. A reactive moiety may then be linked to the carboxylic-acid-terminated precursor polymer.

In some embodiments, an electrophilic moiety may be linked to the carboxylic-acid-terminated precursor polymer. For instance, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated precursor polymer in the presence of a suitable coupling agent (e.g., a carbodiimide coupling agent such as N,N′-dicyclohexylcarbodiimide (DCC), 1-ethyl-3-(3-dimethyl propyl) carbodiimide (EDC), N-hydroxybenzotriazole (HOBt), BOP reagent, and/or another coupling agent) to form a reactive cyclic imide ester (e.g., a succinimide ester group, a maleimide ester group, a glutarimide ester group, a phthalimide ester group, a diglycolimide ester group, a bicyclo[2.2.1]hept-5-ene-2,3-dicarboxylic acid imide ester group, etc.) that is linked to a hydrophilic polymer segment through a hydrolysable ester group. In this way, a number of reactive diester groups can be formed.

For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, and succinimidyl diglycolate groups, among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others. In the particular case of N-hydroxymaleimide as an N-hydroxy cyclic imide compound, exemplary reactive end groups include maleimidyl malonate groups, maleimidyl glutarate groups, maleimidyl succinate groups, maleimidyl adipate groups, and maleimidyl diglycolate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, and phthalimidyl diglycolate groups, among others.

In other aspects of the present disclosure, a system is provided that comprises (a) a first composition that comprises a radiopaque peptide compound as described herein and (b) a second composition that comprises a reactive polymer comprising reactive moieties as described herein, wherein the system is configured to deliver the reactive polymer and the radiopaque peptide compound under conditions such that covalent crosslinks are formed between the reactive polymer and the radiopaque peptide compound.

The first composition may be a first fluid composition comprising the radiopaque peptide compound or a first dry composition that comprises the radiopaque peptide compound, to which a suitable fluid such as water for injection, saline, etc. can be added to form a first fluid composition. In addition to the radiopaque peptide compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer, to which a suitable fluid such as water for injection, saline, etc. can be added to form a second fluid composition. In addition to the reactive polymer, the second composition may further comprise additional agents, including therapeutic agents, imaging agents, colorants, tonicity adjusting agents, suspension agents, wetting agents, and pH adjusting agents as described below.

In some embodiments, the system is configured to combine a first fluid composition comprising the radiopaque peptide compound with a second fluid comprising the reactive polymer. Upon mixing the first and second fluid compositions, the radiopaque peptide compound crosslinks with the reactive polymer, forming a crosslinked product. The first and second fluid compositions may be combined form crosslinked hydrogels, either in vivo or ex vivo.

In some embodiments, the radiopaque peptide compound is initially combined with the reactive polymer under conditions where crosslinking between the reactive polymer and the radiopaque peptide compound is suppressed (e.g., an acidic pH, in some embodiments). Then, when crosslinking is desired, the conditions are changed such that crosslinking is increased (e.g., a change from an acidic pH to a basic pH, in some embodiments), leading to crosslinking between the radiopaque peptide compound and the reactive polymer, thereby forming a crosslinked product.

In some embodiments, the system comprises (a) a first composition that comprises radiopaque peptide compound as described hereinabove, (b) a second composition that comprises a reactive polymer as described hereinabove, and (c) a third composition, specifically, an accelerant composition, that contains an accelerant that is configured to accelerate a crosslinking reaction between the radiopaque peptide compound and the reactive polymer.

The first composition may be a first fluid composition comprising the radiopaque peptide compound that is buffered to an acidic pH or a first dry composition that comprises the radiopaque peptide compound, to which a suitable fluid such as water for injection, saline, an acidic buffer solution, etc. can be added to form a first fluid composition comprising the radiopaque peptide compound that is buffered to an acidic pH. In some embodiments, for example, the acidic buffering composition may comprise monobasic sodium phosphate, among other possibilities. The first fluid composition comprising the radiopaque peptide compound may have a pH ranging, for example, from about 3 to about 5. In addition to the radiopaque peptide compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer from which a fluid composition is formed, for example, by the addition of a suitable fluid such as water for injection, saline, or the first fluid composition comprising the radiopaque peptide compound that is buffered to an acidic pH. In addition to the reactive polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the radiopaque peptide compound that is buffered to an acidic pH and the second composition comprises a dry composition that comprises the reactive polymer. The first composition may then be mixed with the second composition to provide a prepared fluid composition that is buffered to an acidic pH and comprises the radiopaque peptide compound and the reactive polymer. In a particular example, a syringe may be provided that contains the first fluid composition comprising the radiopaque peptide compound that is buffered to an acidic pH, and a vial may be provided that comprises the dry composition (e.g., a powder) that comprises the reactive polymer. The syringe may then be used to inject the first fluid composition into the vial containing the reactive polymer to form a prepared fluid composition that is buffered to an acidic pH and contains the radiopaque peptide compound and the reactive polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11. In addition to the above, the fluid accelerant composition may further comprise additional agents, including those described below.

A prepared fluid composition that is buffered to an acidic pH and comprises the radiopaque peptide compound and the reactive polymer as described above, and a fluid accelerant composition that is buffered to basic pH as described above, may be combined form crosslinked hydrogels, either in vivo or ex vivo.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly(DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethene (BODIPY) analogs, among others, (e) imageable radioisotopes including 99mTc, 201Th, 51Cr, 67Ga, 68Ga, 111 In, 64Cu, 89Zr, 59Fe, 42K, 82Rb, 24Na, 45Ti, 44Sc, 51Cr and 177Lu, among others, and (f) radiocontrast agents, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second compositions to a subject.

In some embodiments, the system may include a delivery device that comprises a first reservoir that contains a first fluid composition that comprises a radiopaque peptide compound as described herein and a second reservoir that contains a second fluid composition that comprises a reactive polymer as described herein, wherein the first and second fluid compositions form a crosslinked product upon mixing. In some embodiments, the system may include a delivery device that comprises a first reservoir that contains a first fluid composition that comprises the radiopaque peptide compound and the reactive polymer and is buffered to first pH, such as the prepared fluid composition previously described, and a second reservoir that contains second fluid composition, for example, a fluid accelerant composition that when combined with the first fluid composition changes a pH of the environment surrounding the radiopaque peptide compound and the reactive polymer (such as the fluid accelerant composition previously described), leading to crosslinking between the radiopaque peptide compound and the reactive polymer.

In either case, during operation, the first fluid composition and second fluid composition are dispensed from the first and second reservoirs and combined, whereupon the radiopaque peptide compound and the reactive polymer and crosslink with one another to form a crosslinked hydrogel.

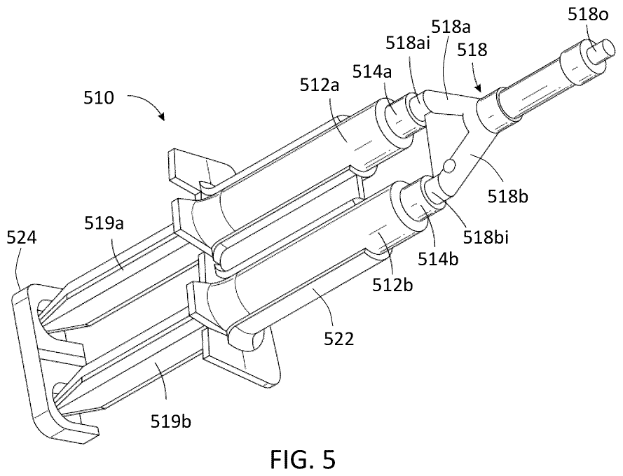

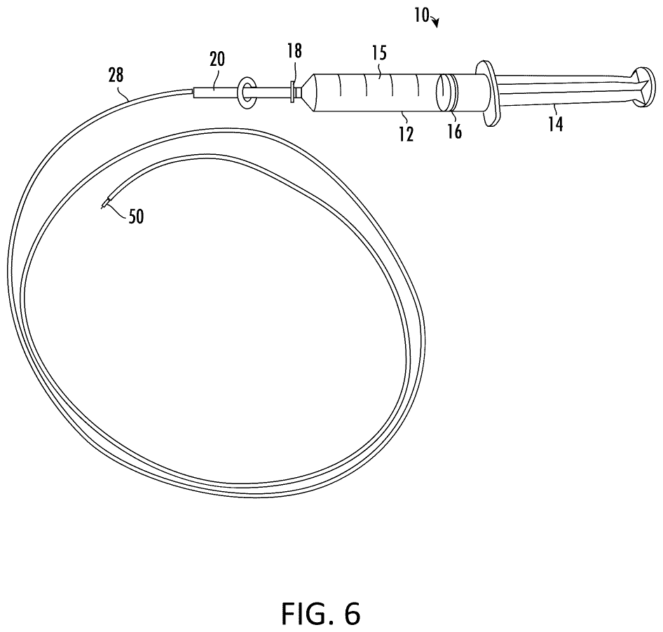

In particular embodiments, and with reference to FIG. 5, the system may include a delivery device 510 that comprises a double-barrel syringe, which includes a first barrel 512a having a first barrel outlet 514a, which first barrel contains a first fluid composition as described above, a first plunger 519a that is movable in the first barrel 512a, a second barrel 512b having a second barrel outlet 514b, which second barrel 512b contains a second fluid composition as described above, and a second plunger 519b that is movable in the second barrel 512b. In some embodiments, the device 510 may further comprise a mixing section 518 (e.g., a Y-connector) having a first mixing section inlet 518ai in fluid communication with the first barrel outlet 514a, a second mixing section inlet 518bi in fluid communication with the second barrel outlet 514b, and a mixing section outlet 5180. Also shown are a syringe holder 522 configured to hold the first and second syringe barrels 512a, 512b, in a fixed relationship and a plunger cap 524 configured to hold the first and second plungers 519a, 519b in a fixed relationship.

In some embodiments, the delivery device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and the radiopaque peptide compound and the reactive polymer ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a crosslinked hydrogel is ultimately formed at the administration location.

After administration, the compositions of the present disclosure can be imaged using a suitable imaging technique. Typically, the imaging technique is an x-ray-based imaging technique, such as computerized tomography or x-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-releasing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Crosslinked hydrogel particles formed using the above and other techniques may vary widely in size, for example, having an average size ranging from 50 to 950 microns.

In addition to a crosslinked hydrogel as described above, crosslinked hydrogel compositions in accordance with the present disclosure may contain additional agents, including therapeutic agents, imaging agents, colorants, tonicity adjusting agents, suspension agents, wetting agents, and pH adjusting agents as described above.

In various embodiments, kits are provided that include one or more delivery devices for delivering the crosslinked hydrogel composition to a subject. Such systems may include one or more of the following: a syringe barrel, which may or may not contain a crosslinked hydrogel composition as described herein; a vial, which may or may not contain a crosslinked hydrogel composition as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel composition may be provided in dry form (e.g., powder form) or in a form that is ready for injection, such as an injectable hydrogel form (e.g., a suspension of crosslinked hydrogel particles).

FIG. illustrates a syringe 10 providing a reservoir for a crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked hydrogel composition 15 for injection through the needle 50.

The crosslinked hydrogel compositions described herein can be used for a number of purposes.

For example, crosslinked hydrogel compositions can be injected to provide spacing between tissues, crosslinked hydrogel compositions can be injected (e.g., in the form of blebs) to provide fiducial markers, crosslinked hydrogel compositions can be injected for tissue augmentation or regeneration, crosslinked hydrogel compositions can be injected as a filler or replacement for soft tissue, crosslinked hydrogel compositions can be injected to provide mechanical support for compromised tissue, crosslinked hydrogel compositions be injected as a scaffold, and/or crosslinked hydrogel compositions can be injected as a carrier of therapeutic agents in the treatment of diseases and cancers and the repair and regeneration of tissue, among other uses.

After administration, the crosslinked hydrogel compositions of the present disclosure can be imaged using a suitable imaging technique.

As seen from the above, the crosslinked hydrogel compositions of the present disclosure may be used in a variety of medical procedures, including the following, among others: a procedure to implant a fiducial marker comprising a crosslinked hydrogel, a procedure to implant a tissue regeneration scaffold comprising a crosslinked hydrogel, a procedure to implant a tissue support comprising a crosslinked hydrogel, a procedure to implant a tissue bulking agent comprising a crosslinked hydrogel, a procedure to implant a therapeutic-agent-containing depot comprising a crosslinked hydrogel, a tissue augmentation procedure comprising implanting a crosslinked hydrogel, a procedure to introduce a crosslinked hydrogel between a first tissue and a second tissue to space the first tissue from the second tissue.

The crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. A method for forming a radiopaque peptide comprising:
(a) performing an amide coupling reaction between (i) a partially protected first iodinated amino acid comprising an unprotected first α-carbon amino group, a protected first α-carbon carboxylic acid group that is protected with a first carboxylic acid protective group and an iodinated aromatic group and (ii) a partially protected first amine-based amino acid comprising an unprotected second α-carbon carboxylic acid group, a protected second α-carbon amino group that is protected with a first amino protective group, and a protected first side chain amino group that is protected with a second amino protective group that differs from the first amino protective group, is deprotected under different conditions from those used to deprotect the first amino protective group, and is stable under conditions used to deprotect the first amino group, thereby forming an amide linkage from the unprotected first α-carbon amino group and the unprotected second α-carbon carboxylic acid group;
(b) deprotecting the protected second α-carbon amino group of the product of step (a) to form a deprotected second α-carbon amino group, while leaving the protected first side chain amino group and the protected first α-carbon carboxylic acid group of the product of step (a) protected;
(c) performing an amide coupling reaction between the deprotected second α-carbon amino group of the product of step (b) and a partially protected third amino acid comprising a protected third α-carbon amino group that is protected with the first amino protective group and an unprotected third α-carbon carboxylic acid group, thereby forming an amide linkage from the deprotected second α-carbon amino group and the unprotected third α-carbon carboxylic acid group;
(d) deprotecting the protected third α-carbon amino group in the product of step (c) to form a deprotected third α-carbon amino group, while leaving all other protected groups in the product of step (c) protected;
(e) optionally repeating steps (c) and (d) with one or more partially protected further amino acids comprising a protected additional α-carbon amino group that is protected with the first amino protective group and an unprotected additional α-carbon carboxylic acid group until a target radiopaque peptide length is achieved; and
(f) after the target radiopaque peptide length is achieved, removing all protective groups.

2. The method of claim 1, wherein the partially protected third amino acid is a partially protected second amine-based amino acid that comprises the protected third α-carbon amino group, the unprotected third α-carbon carboxylic acid group, and a protected second side chain amino group that is protected with the second amino protective group.

3. The method of claim 1, wherein the partially protected third amino acid is a partially protected second iodinated amino acid that comprises the protected third α-carbon amino group, the unprotected third α-carbon carboxylic acid group, and a second iodinated aromatic group.

4. The method of claim 1, wherein the partially protected third amino acid is a partially protected carboxylic-acid-based amino acid that comprises the protected third α-carbon amino group, the unprotected third α-carbon carboxylic acid group, and a side chain carboxylic acid group that is protected with the first carboxylic acid protective group.

5. The method of claim 1, wherein the first carboxylic acid protective group is selected from a C₁-C₆ alkyl group.

6. The method of claim 1, wherein the first amino protective group is an acid labile protecting group.

7. The method of claim 1, wherein the second amino protective group is stable in an acidic environment.

8. The method of claim 1, wherein the first iodinated amino acid is selected from thyroxine, diiodotryosine, and iodophenyl alanine.

9. The method of claim 1, wherein the first amine-based amino acid is selected from lysine and ornithine.

10. The method of claim 1, wherein the target peptide length is between 3 and 20 amino acids.

11. A method for forming a radiopaque peptide comprising:
(a) performing an amide coupling reaction between (i) a partially protected first iodinated amino acid comprising an protected first α-carbon amino group that is protected with a first amino protective group, an unprotected first α-carbon carboxylic acid group and an iodinated aromatic group and (ii) a partially protected first amine-based amino acid comprising a protected second α-carbon carboxylic acid group that is protected with a first carboxylic acid protective group, an unprotected second α-carbon amino group, and a protected first side chain amino group that is protected with a second amino protective group that differs from the first amino protective group, is deprotected under different conditions from those used to deprotect the first amino protective group, and is stable under conditions used to deprotect the first amino group, thereby forming an amide linkage from the unprotected first α-carbon carboxylic acid group and the unprotected second α-carbon amino group;
(b) deprotecting the protected first α-carbon amino group in the product of step (a) to form a deprotected first α-carbon amino group, while leaving all other protected groups in the product of step (a) protected while leaving the protected first side chain amino group and the protected second α-carbon carboxylic acid group of the product of step (a) protected;
(c) performing an amide coupling reaction between the deprotected first α-carbon amino group of the product of step (b) and a partially protected third amino acid comprising a protected third α-carbon amino group that is protected with the first amino protective group and an unprotected third α-carbon carboxylic acid group, thereby forming an amide linkage from the deprotected first α-carbon amino group and the unprotected third α-carbon carboxylic acid group;
(d) deprotecting the protected third α-carbon amino group in the product of step (c) to form a deprotected third α-carbon amino group, while leaving all other protected groups in the product of step (c) protected; and
(e) optionally repeating steps (c) and (d) with one or more partially protected further amino acids comprising a protected additional α-carbon amino group that is protected with the first amino protective group and an unprotected additional α-carbon carboxylic acid group until a target radiopaque peptide length is achieved; and
(f) after the target radiopaque peptide length is achieved, removing all protective groups.

12. The method of claim 11, wherein the partially protected third amino acid is a partially protected second amine-based amino acid that comprises the protected third α-carbon amino group, the unprotected third α-carbon carboxylic acid group, and a protected second side chain amino group that is protected with the second amino protective group.

13. The method of claim 11, wherein the partially protected third amino acid is a partially protected second iodinated amino acid that comprises the protected third α-carbon amino group, the unprotected third α-carbon carboxylic acid group, and a second iodinated aromatic group.

14. The method of claim 11, wherein the partially protected third amino acid is a partially protected carboxylic-acid-based amino acid that comprises the protected third α-carbon amino group, the unprotected third α-carbon carboxylic acid group, and a side chain carboxylic acid group that is protected with the first carboxylic acid protective group.

15. The method of claim 11, wherein the first carboxylic acid protective group is selected from a C₁-C₆ alkyl group.

16. The method of claim 11, wherein the first amino protective group is an acid labile protecting group.

17. The method of claim 11, wherein the second amino protective group is stable in an acidic environment.

18. The method of claim 11, wherein the first iodinated amino acid is selected from thyroxine, diiodotryosine, and iodophenyl alanine.

19. The method of claim 11, wherein the first amine-based amino acid is selected from lysine and ornithine.

20. The method of claim 11, wherein the target peptide length is between 3 and 20 amino acids.
